(19) United States
(12) Patent Application Publication
(10) Pub. No.: US 2024/0144553 A1
(43) Pub. Date: May 2, 2024

(54) SYSTEMS AND METHODS FOR IMAGE RECONSTRUCTION

(71) Applicant: SHANGHAI UNITED IMAGING HEALTHCARE CO., LTD., Shanghai (CN)

(72) Inventors: Tao FENG, Houston, TX (US); Liuchun HE, Shanghai (CN); Aaron SELFRIDGE, Houston, TX (US); Hongdi LI, Houston, TX (US)

(73) Assignee: SHANGHAI UNITED IMAGING HEALTHCARE CO., LTD., Shanghai (CN)

(21) Appl. No.: 18/398,081

(22) Filed: Dec. 27, 2023

Related U.S. Application Data
(63) Continuation of application No. PCT/CN2022/093214, filed on May 17, 2022.

(51) Int. Cl. G06T 11/00; G06T 7/50; G06T 7/70

(52) U.S. Cl. CPC G06T 11/005; G06T 7/50; G06T 7/70; G06T 2207/10104; G06T 2207/30004; G06T 2210/21; G06T 2210/41

(57) ABSTRACT

Systems and methods for image reconstruction are provided. A method may include obtaining image data that includes information of a plurality of coincidence events; for each of the plurality of coincidence events, identifying, based on the information of the coincidence event, identities of two original scintillator elements at each of which a photon of the coincidence event was detected; obtaining a depth of interaction (DOI) of each photon of the coincidence event within one original scintillator element of the two original scintillator elements; and determining, based on the identities and the DOIs of the two original scintillator elements, identities of two corrected scintillator elements each of which corresponds to an original scintillator element of the two original scintillator elements; and generating a reconstructed image based on the identities of two corrected scintillator elements of each of the plurality of coincidence events.

### CROSS-REFERENCE TO RELATED APPLICATIONS

This application is a Continuation of International Application No. PCT/CN2022/093214, filed on May 17, 2022, the contents of which are hereby incorporated by reference.

### TECHNICAL FIELD

The present disclosure generally relates to image reconstruction, and more particularly, relates to systems and methods for positron emission tomography (PET) image reconstruction.

### BACKGROUND

Recently, PET has been widely used in clinical examination and disease diagnosis. In PET, depth of interaction (DOI) information is important for generating high-resolution images and compensating for large angle effects caused by a long axial field of view (FOV). However, using DOI information for image reconstruction is complicated. It is desirable to provide systems and methods for image reconstruction that compensate for DOI effects, thereby improving image resolutions and reducing reconstruction complexity.

### SUMMARY

According to one aspect of the present disclosure, a system for image reconstruction is provided. The system may include at least one storage device including a set of instructions and at least one processor configured to communicate with the at least one storage device. When executing the set of instructions, the at least one processor may be configured to direct the system to perform operations including: obtaining image data that includes information of a plurality of coincidence events; for each of the plurality of coincidence events, identifying, based on the information of the coincidence event, identities of two original scintillator elements at each of which a photon of the coincidence event was detected; obtaining a depth of interaction (DOI) of each photon of the coincidence event within one original scintillator element of the two original scintillator elements; and determining, based on the identities and the DOIs of the two original scintillator elements, identities of two corrected scintillator elements each of which corresponds to an original scintillator element of the two original scintillator elements; and generating a reconstructed image based on the identities of two corrected scintillator elements of each of the plurality of coincidence events.

In some embodiments, for each of the plurality of coincidence events, a corrected DOI of each photon of the coincidence event in the corresponding corrected scintillator element is a pre-determined value.

In some embodiments, the operations may further include: storing the identities of two corrected scintillator elements corresponding to each of the plurality of coincidence events into corrected list-mode data or a corrected sinogram; and generating the reconstructed image based on the corrected list-mode data or the corrected sinogram.

In some embodiments, the determining, based on the identities and the DOIs of the two original scintillator elements, the identities of the two corrected scintillator elements may include: determining, based on the identities and the DOIs of the two original scintillator elements, a projection line of the coincidence event; determining two intersection points of the projection line and a scintillator element array; and determining the identities of the two corrected scintillator elements based on the two intersection points.

In some embodiments, the generating the reconstructed image based on the identities of the two corrected scintillator elements may include: determining a corrected time of flight (TOF) for each of the plurality of coincidence events; and generating the reconstructed image based on the identities of two corrected scintillator elements and the corrected TOF of each of the plurality of coincidence events.

In some embodiments, the determining the corrected TOF may include: identifying an original TOF of the coincidence event; determining, based on the identities and the DOIs of the two original scintillator elements, an estimated time difference between the original TOF and the corrected TOF; and determining the corrected TOF based on the estimated time difference and the original TOF.

In some embodiments, the corrected TOF may be determined according to a TOF simulation algorithm or an experimental TOF result.

In some embodiments, the operations may further include: storing the corrected TOF into corrected list-mode data or a corrected sinogram; and generating the reconstructed image based on the corrected list-mode data or the corrected sinogram.

In some embodiments, the generating the reconstructed image based on the identities of the two corrected scintillator elements of each of the plurality of coincidence events may include: determining, based on the identities of two corrected scintillator elements of each of the plurality of coincidence events, a corrected point-spread-function (PSF) of the coincidence event; and generating the reconstructed image based on the corrected PSF and the identities of two corrected scintillator elements of each of the plurality of coincidence events.

In some embodiments, the corrected PSF may be determined according to a PSF simulation algorithm or an experimental PSF result.

According to another aspect of the present disclosure, a method for image reconstruction is provided. The method may include obtaining image data that includes information of a plurality of coincidence events; for each of the plurality of coincidence events, identifying, based on the information of the coincidence event, identities of two original scintillator elements at each of which a photon of the coincidence event was detected; obtaining a depth of interaction (DOI) of each photon of the coincidence event within one original scintillator element of the two original scintillator elements; and determining, based on the identities and the DOIs of the two original scintillator elements, identities of two corrected scintillator elements each of which corresponds to an original scintillator element of the two original scintillator elements; and generating a reconstructed image based on the identities of two corrected scintillator elements of each of the plurality of coincidence events.

According to another aspect of the present disclosure, a non-transitory computer-readable storage medium including instructions is provided. When accessed by at least one processor of a system for image reconstruction, the instructions cause the system to perform a method. The method may include obtaining image data that includes information of a plurality of coincidence events; for each of the plurality of coincidence events, identifying, based on the information of the coincidence event, identities of two original scintillator elements at each of which a photon of the coincidence event was detected; obtaining a depth of interaction (DOI) of each photon of the coincidence event within one original scintillator element of the two original scintillator elements; and determining, based on the identities and the DOIs of the two original scintillator elements, identities of two corrected scintillator elements each of which corresponds to an original scintillator element of the two original scintillator elements; and generating a reconstructed image based on the identities of two corrected scintillator elements of each of the plurality of coincidence events.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2 is a schematic diagram illustrating exemplary hardware and/or software components of a computing device according to some embodiments of the present disclosure;

FIG. 3 is a schematic diagram illustrating exemplary hardware and/or software components of a mobile device according to some embodiments of the present disclosure;

FIG. 4 is a block diagram illustrating an exemplary processing device according to some embodiments of the present disclosure;

FIG. 5 is a flowchart illustrating an exemplary process for image reconstruction according to some embodiments of the present disclosure;

FIG. 6 is a schematic diagram illustrating an exemplary detector along a transverse direction and an axial direction according to some embodiments of the present disclosure;

FIG. 7 illustrates exemplary reconstructed images of a plurality of rods according to some embodiments of the present disclosure;

FIG. 8 is a flowchart illustrating an exemplary process for determining a corrected TOF according to some embodiments of the present disclosure;

FIG. 9 is a flowchart illustrating an exemplary process for generating a reconstructed image according to some embodiments of the present disclosure;

FIG. 10A illustrates exemplary corrected PSFs according to some embodiments of the present disclosure;

FIG. 10B illustrates exemplary original PSFs according to some embodiments of the present disclosure;

FIG. 11A illustrates FWHMs of exemplary corrected PSFs and exemplary original PSFs along a transverse direction according to some embodiments of the present disclosure;

FIG. 11B illustrates FWHMs of exemplary corrected PSFs along an axial direction according to some embodiments of the present disclosure; and

FIG. 11C illustrates FWHMs of exemplary original PSFs along an axial direction according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

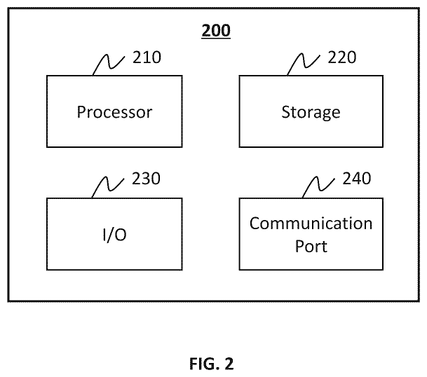

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 210 as illustrated in FIG. 2) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “image” in the present disclosure is used to collectively refer to image data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Provided herein are systems and methods for non-invasive biomedical imaging, such as for disease diagnostic or research purposes. In some embodiments, the systems may include a single modality imaging system and/or a multi-modality imaging system. The single modality imaging system may include, for example, a positron emission tomography (PET) system. The multi-modality imaging system may include, for example, a positron emission tomography-computed tomography (PET-CT) system, a positron emission tomography-magnetic resonance imaging (PET-MRI) system. It should be noted that the imaging system described below is merely provided for illustration purposes, and not intended to limit the scope of the present disclosure.

The term “imaging modality” or “modality” as used herein broadly refers to an imaging method or technology that gathers, generates, processes, and/or analyzes imaging information of a subject. The subject may include a biological object and/or a non-biological object. The biological object may be a human being, an animal, a plant, or a portion thereof (e.g., a cell, a tissue, an organ, etc.). In some embodiments, the subject may be a man-made composition of organic and/or inorganic matters that are with or without life.

An aspect of the present disclosure relates to systems and methods for image reconstruction. The systems and methods may obtain image data including information of a plurality of coincidence events. For each of the plurality of coincidence events, the systems and methods may identify, based on the information of a plurality of coincidence events, identities of two original scintillator elements at each of which a photon of the coincidence event was detected, and obtain a depth of interaction (DOI) of each photon of the coincidence event within one original scintillator element of the two original scintillator elements. The systems and methods may determine, based on the identities and the DOIs of the two original scintillator elements, identities of two corrected scintillator elements each of which corresponds to an original scintillator element of the two original scintillator elements. In some embodiments, a corrected DOI of each photon of the coincidence event in the corresponding corrected scintillator element may be a predetermined value (e.g., 0, 1, 2, 3, etc.). The systems and methods may generate a reconstructed image based on the identities of two corrected scintillator elements of each of the plurality of coincidence events. In some embodiments, the identities and/or the corrected DOIs of the two corrected scintillator elements corresponding to each of the plurality of coincidence events may be stored in corrected list-mode data or a corrected sinogram, and the systems and methods may generate the reconstructed image based on the corrected list-mode data or the corrected sinogram. In this way, DOI effects may be compensated for in the list-mode data or the sinogram, thereby improving image quality while obliviating the need to modify an existing reconstruction algorithm.

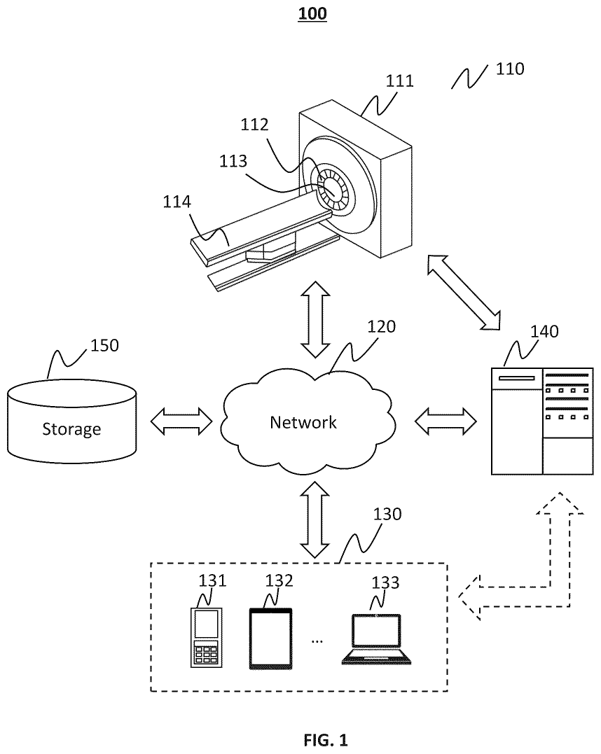

FIG. 1 is a schematic diagram illustrating an exemplary imaging system 100 according to some embodiments of the present disclosure. As shown, the imaging system 100 may include a scanner 110, a network 120, one or more terminals 130, a processing device 140, and a storage device 150. In some embodiments, the scanner 110, the terminal(s) 130, the processing device 140, and/or the storage device 150 may be connected to and/or communicate with each other via a wireless connection (e.g., the network 120), a wired connection, or a combination thereof. The connection between the components of the imaging system 100 may be variable. Merely by way of example, the scanner 110 may be connected to the processing device 140 through the network 120, as illustrated in FIG. 1. As another example, the scanner 110 may be connected to the processing device 140 directly. As a further example, the storage device 150 may be connected to the processing device 140 through the network 120, as illustrated in FIG. 1, or connected to the processing device 140 directly. As still a further example, a terminal 130 may be connected to the processing device 140 through the network 120, as illustrated in FIG. 1, or connected to the processing device 140 directly.

The scanner 110 may generate or provide image data related to a subject via scanning the subject. In some embodiments, the subject may include a biological object and/or a non-biological object. For example, the subject may include a specific portion of a body, such as a head, a thorax, an abdomen, or the like, or a combination thereof. In some embodiments, the scanner 110 may include a single-modality scanner (e.g., a PET scanner) and/or multi-modality scanner (e.g., a PET-CT scanner, a PET-MRI scanner) as described elsewhere in this disclosure.

In some embodiments, the scanner 110 may include a gantry 111, a detector 112, a detecting region 113, and a scanning table 114. The gantry 111 may support the detector 112. The detector 112 may detect coincidence events emitted from the detection region 113. In some embodiments, the detector 112 may include one or more detector units. The detector units may be assembled in any suitable manner, for example, a ring, an arc, a rectangle, an array, or the like, or any combination thereof. In some embodiments, a detector unit may include one or more scintillator elements (e.g., scintillators) and/or one or more photomultipliers (e.g., silicon photomultiplier (SiPM), photomultiplier tube (PMT)). The one or more scintillator elements may be arranged in a scintillator element array. The detector 112 may include a plurality of scintillator element arrays arranged in a certain structure (e.g., a polygon barrel structure, a hemispherical structure, etc. The scanning table 114 may transport the subject into and out of, and facilitate the positioning of the subject in the detection region 113. In some embodiments, the detected coincidence events may be stored or archived in a storage device (e.g., the storage device 150), displayed on a display, or transferred to an external storage device via a cable, or a wired or wireless network (e.g., the network 120). In some embodiments, a user may control the scanner 110 via the processing device 140 and/or the terminal(s) 130.

In some embodiments, the scanner 110 may be a PET scanner. Before scanning, a radioactive tracer isotope may be injected into the subject to be scanned. One or more atoms of the tracer isotope may be chemically incorporated into biologically active molecules in the subject. The active molecules may become concentrated in a tissue of interest within the subject. The tracer isotope may undergo positron emission decay and emit positrons. A positron may travel a short distance (e.g., about 1 mm) within a tissue of interest, lose kinetic energy, and interact with an electron of the subject. The positron and the electron may annihilate and produce a pair of annihilation photons. The pair of annihilation photons (or radiation rays) may move in approximately opposite directions. A plurality of radiation rays may reach the detector 112 and be detected by the detector 112.

In some embodiments, one or more coincidence events may be determined based on the interaction positions and the interaction times of a plurality of received photons. If two photons are received and interact with two scintillator elements of two detector units within a certain coincidence time window (e.g., 1 nanosecond, 2 nanoseconds, 5 nanoseconds, 10 nanoseconds, 20 nanoseconds, etc.), the two photons may be deemed to come from a same annihilation, and regarded as a coincidence event (or coincident event).

The network 120 may include any suitable network that can facilitate the exchange of information and/or data for the imaging system 100. In some embodiments, one or more components of the imaging system 100 (e.g., the scanner 110, the processing device 140, the storage device 150, the terminal(s) 130) may communicate information and/or data with one or more other components of the imaging system 100 via the network 120. For example, the processing device 140 may obtain image data of a subject from the scanner 110 via the network 120. As another example, the processing device 140 may obtain user instruction(s) from the terminal(s) 130 via the network 120. The network 120 may be or include a public network (e.g., the Internet), a private network (e.g., a local area network (LAN)), a wired network, a wireless network (e.g., an 802.11 network, a Wi-Fi network), a frame relay network, a virtual private network (VPN), a satellite network, a telephone network, routers, hubs, switches, server computers, and/or any combination thereof. For example, the network 120 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 120 may include one or more network access points. For example, the network 120 may include wired and/or wireless network access points such as base stations and/or internet exchange points through which one or more components of the imaging system 100 may be connected to the network 120 to exchange data and/or information.

The terminal(s) 130 may be connected to and/or communicate with the scanner 110, the processing device 140, and/or the storage device 150. For example, the terminal(s) 130 may display a reconstructed image of the subject. In some embodiments, the terminal(s) 130 may include a mobile device 131, a tablet computer 132, a laptop computer 133, or the like, or any combination thereof. For example, the mobile device 131 may include a mobile phone, a personal digital assistant (PDA), a gaming device, a navigation device, a point of sale (POS) device, a laptop, a tablet computer, a desktop, or the like, or any combination thereof. In some embodiments, the terminal(s) 130 may include an input device, an output device, etc. In some embodiments, the terminal(s) 130 may be part of the processing device 140.

The processing device 140 may process data and/or information obtained from the scanner 110, the storage device 150, the terminal(s) 130, or other components of the imaging system 100. For example, the processing device 140 may obtain image data that includes information of a plurality of coincidence events. For each of the plurality of coincidence events, the processing device 140 may identify identities of two original scintillator elements at each of which a photon of the coincidence event was detected and a depth of interaction (DOI) of each photon of the coincidence event within one original scintillator element of the two original scintillator elements. The processing device 140 may determine, based on the identities and the DOIs of the two original scintillator elements, identities of two corrected scintillator elements each of which corresponds to an original scintillator element of the two original scintillator elements. As another example, the processing device 140 may generate a reconstructed image based on the identities of two corrected scintillator elements of each of the plurality of coincidence events. As still another example, the processing device 140 may further determine a corrected time of flight (TOF), and generate the reconstructed image based on the identities of two corrected scintillator elements and the corrected TOF of each of the plurality of coincidence events.

In some embodiments, the processing device 140 may be a single server or a server group. The server group may be centralized or distributed. In some embodiments, the processing device 140 may be local to or remote from the imaging system 100. For example, the processing device 140 may access information and/or data from the scanner 110, the storage device 150, and/or the terminal(s) 130 via the network 120. As another example, the processing device 140 may be directly connected to the scanner 110, the terminal(s) 130, and/or the storage device 150 to access information and/or data. In some embodiments, the processing device 140 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 140 may be implemented by a computing device 200 having one or more components as described in connection with FIG. 2.

In some embodiments, the processing device 140 may include one or more processors (e.g., single-core processor(s) or multi-core processor(s)). Merely by way of example, the processing device 140 may include a central processing unit (CPU), an application-specific integrated circuit (ASIC), an application-specific instruction-set processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction-set computer (RISC), a microprocessor, or the like, or any combination thereof.

The storage device 150 may store data, instructions, and/or any other information. In some embodiments, the storage device 150 may store data obtained from the processing device 140, the terminal(s) 130, and/or the scanner 110. In some embodiments, the storage device 150 may store data and/or instructions that the processing device 140 may execute or use to perform exemplary methods described in the present disclosure. In some embodiments, the storage device 150 may include a mass storage device, a removable storage device, a volatile read-and-write memory, a read-only memory (ROM), or the like, or any combination thereof. Exemplary mass storage devices may include a magnetic disk, an optical disk, a solid-state drive, etc. Exemplary removable storage devices may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 150 may be implemented on a cloud platform as described elsewhere in the disclosure.

In some embodiments, the storage device 150 may be connected to the network 120 to communicate with one or more other components of the imaging system 100 (e.g., the processing device 140, the terminal(s) 130). One or more components of the imaging system 100 may access the data or instructions stored in the storage device 150 via the network 120. In some embodiments, the storage device 150 may be part of the processing device 140.

It should be noted that the above description of the imaging system 100 is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. For example, the imaging system 100 may include one or more additional components. Additionally or alternatively, one or more components of the imaging system 100 described above may be omitted. As another example, two or more components of the imaging system 100 may be integrated into a single component.

FIG. 2 is a schematic diagram illustrating exemplary hardware and/or software components of a computing device 200 according to some embodiments of the present disclosure. The computing device 200 may be used to implement any component of the imaging system 100 as described herein. For example, the processing device 140 and/or the terminal(s) 130 may be implemented on the computing device 200, respectively, via its hardware, software program, firmware, or a combination thereof. Although only one such computing device is shown, for convenience, the computer functions relating to the imaging system 100 as described herein may be implemented in a distributed fashion on a number of similar platforms, to distribute the processing load. As illustrated in FIG. 2, the computing device 200 may include a processor 210, a storage device 220, an input/output (I/O) 230, and a communication port 240.

The processor 210 may execute computer instructions (e.g., program code) and perform functions of the processing device 140 in accordance with techniques described herein. The computer instructions may include, for example, routines, programs, objects, components, data structures, procedures, modules, and functions, which perform particular functions described herein. For example, the processor 210 may process image data obtained from the scanner 110, the terminal(s) 130, the storage device 150, and/or any other component of the imaging system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors, thus operations and/or method operations that are performed by one processor as described in the present disclosure may also be jointly or separately performed by the multiple processors. For example, if in the present disclosure the processor of the computing device 200 executes both operation A and operation B, it should be understood that operation A and operation B may also be performed by two or more different processors jointly or separately in the computing device 200 (e.g., a first processor executes operation A and a second processor executes operation B, or the first and second processors jointly execute operations A and B).

The storage device 220 may store data/information obtained from the scanner 110, the terminal(s) 130, the storage device 150, and/or any other component of the imaging system 100. In some embodiments, the storage device 220 may include a mass storage device, a removable storage device, a volatile read-and-write memory, a read-only memory (ROM), or the like, or any combination thereof. In some embodiments, the storage device 220 may store one or more programs and/or instructions to perform exemplary methods described in the present disclosure. For example, the storage device 220 may store a program for the processing device 140 to execute to generate a scatter sinogram generator.

The I/O 230 may input and/or output signals, data, information, etc. In some embodiments, the I/O 230 may enable a user interaction with the processing device 140. In some embodiments, the I/O 230 may include an input device and an output device. The input device may include alphanumeric and other keys that may be input via a keyboard, a touch screen (for example, with haptics or tactile feedback), a speech input, an eye tracking input, a brain monitoring system, or any other comparable input mechanism. The input information received through the input device may be transmitted to another component (e.g., the processing device 140) via, for example, a bus, for further processing. Other types of the input device may include a cursor control device, such as a mouse, a trackball, or cursor direction keys, etc. The output device may include a display (e.g., a liquid crystal display (LCD), a light-emitting diode (LED)-based display, a flat panel display, a curved screen, a television device, a cathode ray tube (CRT), or a touch screen), a speaker, a printer, or the like, or a combination thereof.

The communication port 240 may be connected to a network (e.g., the network 120) to facilitate data communications. The communication port 240 may establish connections between the processing device 140 and the scanner 110, the terminal(s) 130, and/or the storage device 150. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBeen™ link, a mobile network link (e.g., 3G, 4G, 5G), or the like, or a combination thereof. In some embodiments, the communication port 240 may be and/or include a standardized communication port, such as RS232, RS485, etc. In some embodiments, the communication port 240 may be a specially designed communication port. For example, the communication port 240 may be designed in accordance with the digital imaging and communications in medicine (DICOM) protocol.

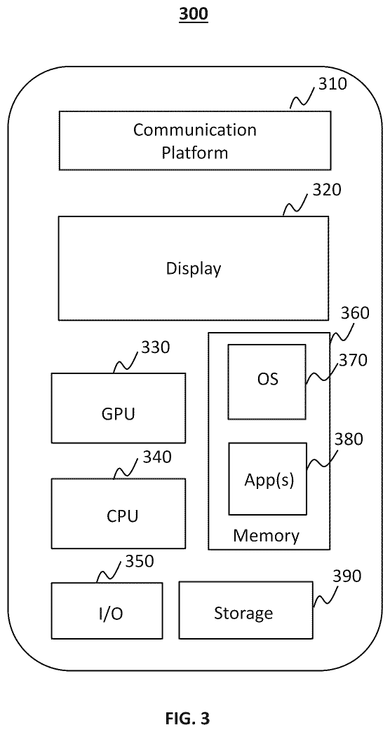

FIG. 3 is a schematic diagram illustrating exemplary hardware and/or software components of a mobile device 300 according to some embodiments of the present disclosure. In some embodiments, one or more components (e.g., a terminal 130 and/or the processing device 140) of the imaging system 100 may be implemented on the mobile device 300.

As illustrated in FIG. 3, the mobile device 300 may include a communication platform 310, a display 320, a graphics processing unit (GPU) 330, a central processing unit (CPU) 340, an I/O 350, a memory 360, and storage 390. In some embodiments, any other suitable component, including but not limited to a system bus or a controller (not shown), may also be included in the mobile device 300. In some embodiments, a mobile operating system 370 (e.g., iOS™, Android™, Windows Phone™) and one or more applications 380 may be loaded into the memory 360 from the storage 390 in order to be executed by the CPU 340. The applications 380 may include a browser or any other suitable mobile apps for receiving and rendering information relating to image processing or other information from the processing device 140. User interactions with the information stream may be achieved via the I/O 350 and provided to the processing device 140 and/or other components of the imaging system 100 via the network 120.

To implement various modules, units, and their functionalities described in the present disclosure, computer hardware platforms may be used as the hardware platform(s) for one or more of the elements described herein. A computer with user interface elements may be used to implement a personal computer (PC) or any other type of work station or terminal device. A computer may also act as a server if appropriately programmed.

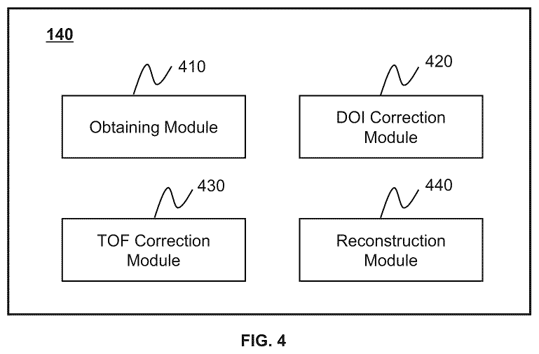

FIG. 4 is a block diagram illustrating an exemplary processing device 140 according to some embodiments of the present disclosure. As shown in FIG. 4, the processing device 140 may include an obtaining module 410, a DOI correction module 420, a TOF correction module 430, and a reconstruction module 440.

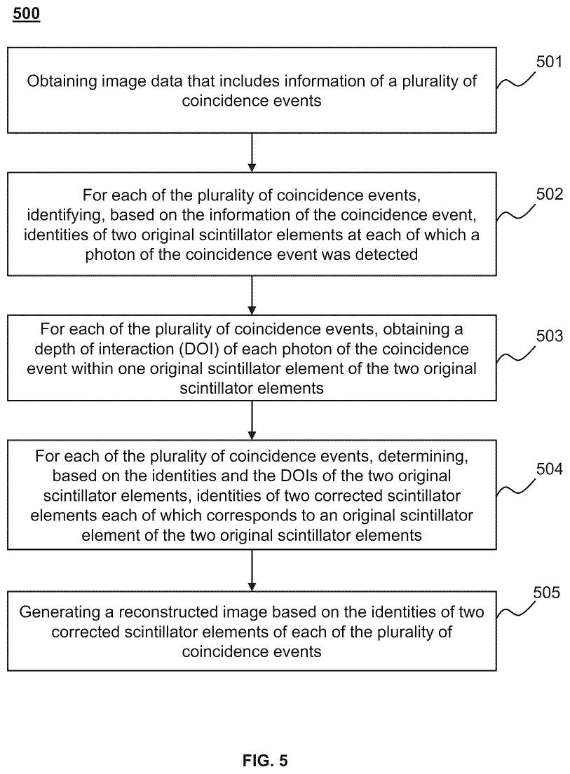

The obtaining module 410 may be configured to obtain image data that includes information of a plurality of coincidence events. More descriptions regarding the obtaining of the image data may be found elsewhere in the present disclosure. See, e.g., operation 501 in FIG. 5 and relevant descriptions thereof.

The DOI correction module 420 may be configured to correct DOI effects. For example, for each of the plurality of coincidence events, the DOI correction module 420 may identify, based on the information of the coincidence event, identities of two original scintillator elements at each of which a photon of the coincidence event was detected. As another example, for each of the plurality of coincidence events, the DOI correction module 420 may obtain a DOI of each photon of the coincidence event within one original scintillator element of the two original scintillator elements. As still another example, for each of the plurality of coincidence events, the DOI correction module 420 may determine, based on the identities and the DOIs of the two original scintillator elements, identities of two corrected scintillator elements each of which corresponds to an original scintillator element of the two original scintillator elements. More descriptions regarding the correcting DOI effects may be found elsewhere in the present disclosure. See, e.g., operations 502-504 in FIG. 5 and relevant descriptions thereof.

The TOF correction module 430 may be configured to determine a corrected TOF. For example, for each of the plurality of coincidence events, the TOF correction module 430 may identify an original TOF of the coincidence event. As another example, the TOF correction module 430 may determine, based on the identities and the DOIs of the two original scintillator elements, an estimated time difference between the original TOF and the corrected TOF. As still another example, the TOF correction module 430 may determine the corrected TOF based on the estimated time difference and the original TOF. More descriptions regarding the determining the corrected TOF may be found elsewhere in the present disclosure. See, e.g., operations 801-803 in FIG. 5 and relevant descriptions thereof.

The reconstruction module 440 may be configured to generate a reconstructed image based on the identities of two corrected scintillator elements of each of the plurality of coincidence events. For example, the reconstruction module 440 may determine, based on the identities of two corrected scintillator elements of each of the plurality of coincidence events, a corrected point-spread-function (PSF) of the coincidence event. As another example, the reconstruction module 440 may generate the reconstructed image based on the corrected PSF and the identities of two corrected scintillator elements of each of the plurality of coincidence events.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, any one of the modules may be divided into two or more units. For instance, the DOI correction module 420 may be divided into two or more units. In some embodiments, two or more modules may be integrated into one module. For example, the DOI correction module 420 and the TOF correction module 430 may be integrated into a correction module.

FIG. 5 is a flowchart illustrating an exemplary process 500 for image reconstruction according to some embodiments of the present disclosure. In some embodiments, the process 500 may be executed by the imaging system 100. For example, the process 500 may be implemented as a set of instructions (e.g., an application) stored in a storage device (e.g., the storage device 150, the storage device 220, and/or the storage 390). In some embodiments, the processing device 140 (e.g., the processor 210 of the computing device 200, the CPU 340 of the mobile device 300, and/or one or more modules illustrated in FIG. 4) may execute the set of instructions and may accordingly be directed to perform the process 500.

In 501, the processing device 140 (e.g., the obtaining module 410) may obtain image data that includes information of a plurality of coincidence events.

In some embodiments, the plurality of coincidence events may have occurred and been detected in a PET scan of a subject. As used herein, the subject may be biological or non-biological. For example, the subject may include a patient (or a portion thereof), an animal, a man-made object (e.g., a phantom), etc., as described elsewhere in the present disclosure (e.g., FIG. 1 and the descriptions thereof). In some embodiments, information of a coincidence event may record characteristics of two photons interacting with the detector 112 (e.g., the scintillator element arrays, the one or more scintillator elements). For example, the information of a coincidence event may include an identity (or a location) of each scintillator element (also referred to as an original scintillator element) that has detected one of the two photons of the coincidence event, a depth of interaction (DOI, also referred to as an original DOI) of each photon, a detected time point of each photon, a time of flight (TOF, also referred to as an original TOF) of the coincidence event, an energy of each photon, or the like, or any combination thereof. In some embodiments, the detector 112 may include a plurality of scintillator element arrays, and a plurality of scintillator elements may be arranged in each scintillator element array. In some embodiments, the plurality of scintillator element arrays may be arranged in a circumferential direction to form a polygon barrel structure (or a hemispherical structure). The identity (or the location) of a scintillator element that has detected one of the two photons of a coincident event may indicate which scintillator element of the scintillator element arrays has detected the photon. In some embodiments, the DOI may be the depth inside a scintillator element that has interacted with a photon so as to produce an optical signal. In some embodiments, the TOF of a coincidence event may refer to the time difference between two time points when the two photons of the coincidence event are detected. In some embodiments, the image data that includes information of the plurality of coincidence events may be stored in a format that is suitable for image reconstruction. For example, the image data may be stored in the form of list-mode data, a sinogram, a histogram, or the like, or any combination thereof.

In some embodiments, a scanner (e.g., a PET scanner, a PET-CT scanner, a PET-MR scanner) may be directed to perform a PET scan on the subject to acquire the image data. The processing device 140 may obtain the image data of the subject from the scanner. As another example, the image data may be previously acquired and stored in a storage device (e.g., the storage device 150, the storage device 220, and/or the storage 390). The processing device 140 may obtain the image data of the subject from the storage device via a network (e.g., the network 120).

In 502, for each of the plurality of coincidence events, the processing device 140 (e.g., the DOI correction module 420) may identify, based on the information of the coincidence event, identities of two original scintillator elements at each of which a photon of the coincidence event was detected.

As used herein, an original scintillator element with respect to a photon of a coincident event (or referred as to an original scintillator element for brevity) refers to a scintillator element that has interacted with the photon of the coincidence event so as to generate an optical signal. An identity of the original scintillator element may indicate which scintillator element in the scintillator element arrays (or the detector 112) has detected the photon. In some embodiments, the processing device 140 may retrieve the information of the plurality of coincidence events. For each of the plurality of coincidence events, the processing device 140 may identify, from the information of the coincidence event, the identities of two original scintillator elements at each of which a photon of the coincidence event has been detected.

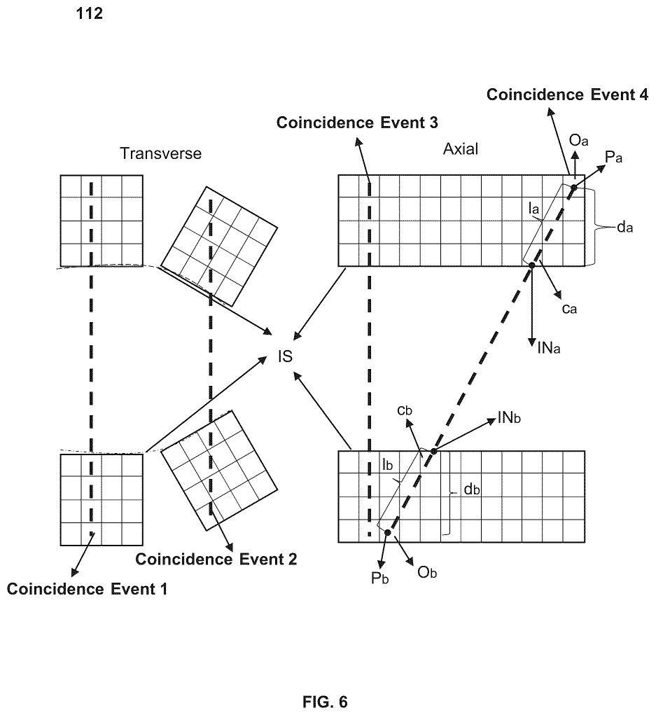

FIG. 6 is a schematic diagram illustrating an exemplary detector 112 along a transverse direction and an axial direction according to some embodiments of the present disclosure. As shown in FIG. 6, scintillator element arrays of the detector 112 may include a plurality of scintillator elements. Coincidence Event 1, Coincidence Event 2, Coincidence Event 3, and Coincidence Event 4 were detected by the detector 112. For example, for Coincidence Event 4, two photons including a first photon and a second photon were detected by the detector 112. An identity of a first original scintillator element that detected the first photon is Oa, and an identity of a second original scintillator element that detected the second photon is Ob. Pa indicates the location where the first photon Oa was detected within the first original scintillator element. Pb indicates the location where the second photon Ob was detected within the second original scintillator element. INa indicates the location on the photon incident surface IS of the scintillator element arrays where the first photon Oa entered the scintillator element arrays before interacting with the first original scintillator element of the scintillator element arrays. INb indicates the location on the photon incident surface IS of the scintillator element arrays where the second photon Ob entered the scintillator element arrays before interacting with the second original scintillator element of the scintillator element arrays.

In 503, for each of the plurality of coincidence events, the processing device 140 (e.g., the DOI correction module 420) may obtain a DOI of each photon of the coincidence event within one original scintillator element of the two original scintillator elements.

As used herein, a DOI of a photon of a coincident event (or referred as to a DOI for brevity) refers to the depth inside the scintillator element arrays (or the detector 112) where the photon has interacted with a scintillator element of the scintillator element arrays (or the detector 112) so as to produce an optical signal. For example, the DOI may be a distance between a location where the interaction between a photon and the original scintillator element has occurred and a photon incident surface of the scintillator element arrays to which the scintillator element belongs. As used herein, a photon incident surface of the scintillator element arrays (of a detector) with the shape of a bore (e.g., a hollow cylinder) refers to an inner surface of the bore (e.g., the inner surface of the hollow cylinder) where a photon impinges on and enters the scintillator element arrays. As shown in FIG. 6, for Coincidence Event 4, a DOI of the first photon is da, and a DOI of the second photon is db. In some embodiments, the processing device 140 may determine the DOI of each photon of a coincidence event based on the information of the coincidence event. In some embodiments, the processing device 140 may retrieve the information of the coincidence event to obtain the DOI of the coincidence event.

In 504, for each of the plurality of coincidence events, the processing device 140 (e.g., the DOI correction module 420) may determine, based on the identities and the DOIs of the two original scintillator elements, identities of two corrected scintillator elements each of which corresponds to an original scintillator element of the two original scintillator elements.

As used herein, a corrected scintillator element with respect to a photon of a coincident event (or referred as to a corrected scintillator element for brevity) refers to a scintillator element determined based on an original scintillator element with respect to the photon by converting the DOI of the photon interacting with the original scintillator element to a corrected DOI. By involving corrected DOIs, the complexity of image reconstruction due to the DOIs may be reduced. In some embodiments, for each of the plurality of coincidence events, the corrected DOI of each photon of the coincidence event in the corresponding corrected scintillator element may be a predetermined value. For example, the predetermined value may be 0, that is, the corrected scintillator element may be a scintillator element on the photon incident surface of the scintillator element arrays. In some embodiments, the predetermined value may be 2 or 3 different values to improve the accuracy of the spatial resolution of a reconstructed image. The predetermined value may be determined based on a location of the original scintillator element. For example, if the original scintillator element is located at a first layer type (e.g., from a 1^{st} layer of the scintillator element arrays to a 8^{th} layer of the scintillator element arrays), the predetermined value may be 0, that is, the corrected scintillator element may be a scintillator element on the photon incident surface of the scintillator element arrays (or a scintillator element at the 1^{st} layer of the scintillator element arrays). As another example, if the original scintillator element is located at a second layer type (e.g., from a 9^{th} layer of the scintillator element arrays to a 16^{th} layer of the scintillator element arrays), the predetermined value may be 2, that is, the corrected scintillator element may be a scintillator element at the 3^{rd} layer of the scintillator element arrays. In some embodiments, the predetermined values may be predetermined and stored in a storage device (e.g., the storage device 150, the storage device 220, and/or the storage 390).

In some embodiments, to determine the identities of the two corrected scintillator elements of each coincidence event, the processing device 140 may determine, based on the identities and the DOIs of the two original scintillator elements, a projection line of the coincidence event. The projection line of the coincident event, also referred to as a line of response (LOR), may be a line connecting two locations (or scintillator elements) where two photons of the coincidence event have been detected. In some embodiments, the processing device 140 may determine, based on the identity of one original scintillator element that has detected a photon of the coincidence event and the DOI of the photon, a location within the original scintillator element where the photon has interacted with the original scintillator element. For example, as shown in FIG. 6, the processing device 140 may determine a location Pa in the first original scintillator element where the first photon interacted with the first original scintillator element based on the identity Oa and the DOI da of the first photon. Techniques for identifying DOI of a photon within a scintillator element or a scintillator element array may be found in, e.g., U.S. Pat. No. 9,645,252B2, CN103901463B, the contents of each of which are hereby incorporated by reference. The processing device 140 may determine a location Pb in the second original scintillator element where the second photon interacted with the second original scintillator element based on the identity Ob and the DOI db of the second photon. The processing device 140 may determine a line between the location Pa and the location Pb as a projection line PaPb of Coincidence Event 4. In some embodiments, the processing device 140 may determine two intersection points of the projection line and the photon incident surface of the scintillator element arrays. For example, as shown in FIG. 6, the projection line PaPb of Coincidence Event 4 intersects with the photon incident surface IS of the detector 112 (or the scintillator element arrays) at points INa and INb. The processing device 140 may determine the points INa and INb as the two intersection points of the projection line and the scintillator element arrays. In some embodiments, the processing device 140 may determine the identities of the two corrected scintillator elements based on the two intersection points. For example, the processing device 140 may determine two scintillator elements of the scintillator element arrays at which the two intersection points INa and INb are located as the two corrected scintillator elements, and identify the identities of the two corrected scintillator elements. As illustrated in FIG. 6, the processing device 140 may identify that an identity of a first corrected scintillator element at which the intersection point INa is located is Ca, and an identity of a second corrected scintillator element at which the intersection point INb is located is Cb. The first corrected scintillator element Ca corresponds to the first original scintillator element Oa, and the second corrected scintillator element Cb corresponds to the second original scintillator element Ob.

In 505, the processing device 140 (e.g., the reconstruction module 440) may generate a reconstructed image based on the identities of two corrected scintillator elements of each of the plurality of coincidence events.

In some embodiments, the processing device 140 may store the identities of two corrected scintillator elements corresponding to each of the plurality of coincidence events and/or the corrected DOIs of each of the plurality of coincidence events in a format that is suitable for image reconstruction. For example, the identities of two corrected scintillator elements corresponding to each of the plurality of coincidence events and/or the corrected DOIs of each of the plurality of coincidence events may be stored in the form of list-mode data (or referred to as corrected list-mode data), a sinogram (or referred to as a corrected sinogram), a histogram (or referred to as a corrected histogram), or the like, or any combination thereof. For example, the processing device 140 may replace an identity of an original scintillator element of each coincidence event with an identity of a corrected scintillator element corresponding to the original scintillator element in original list-mode data (or an original sinogram, an original histogram), and further replace a DOI of a photon of each coincidence event with a corrected DOI (e.g., 0) of the photon in original list-mode data (or an original sinogram, an original histogram) to obtain the corrected list-mode data (or the corrected sinogram, the corrected histogram). As another example, the processing device 140 may only replace an identity of an original scintillator element of each coincidence event with an identity of a corrected scintillator element corresponding to the original scintillator element in original list-mode data (or an original sinogram, an original histogram) to obtain the corrected list-mode data (or the corrected sinogram, the original histogram); in the corrected list-mode data (or the corrected sinogram, the original histogram), the DOIs may be 0. In some embodiments, the processing device 140 may generate the reconstructed image based on the corrected list-mode data (or the corrected sinogram, the corrected histogram). For example, the processing device 140 may generate the reconstructed image based on the corrected list-mode data (or the corrected sinogram, the corrected histogram) according to a reconstruction algorithm. Exemplary reconstruction algorithms may include an expectation maximization (EM) algorithm, a maximum likelihood expectation maximization (MLEM) algorithm, a complete ordered subset expectation maximization (C-OSEM) algorithm, a filter back projection (FBP) algorithm, or the like, or any combination thereof. By maintaining the data structure of the original list-mode data (or the original sinogram, the original histogram) in the corrected list-mode data (or the corrected sinogram, the corrected histogram), no modification may be needed with respect to the reconstruction algorithm for generating the reconstructed image.

In some embodiments, for each of the plurality of coincidence events, the processing device 140 may further determine a corrected time of flight (TOF). The corrected TOF may be used to correct TOF differences caused by using the corrected scintillator elements, instead of the original scintillator elements. In some embodiments, the processing device 140 may perform one or more operations in process 800 as described in connection with FIG. 8 to determine the corrected TOF of each coincidence event.

In some embodiments, the processing device 140 may determine the corrected TOF of each coincidence event according to a TOF simulation algorithm. For example, the processing device 140 may determine the corrected TOF of each coincidence event based on the identities of the two corrected scintillator elements of the coincidence event by applying a Monte-Carlo Simulation algorithm. For example, the processing device 140 may utilize a Monte-Carlo simulation tool (e.g., a software, an application, a toolkit) to simulate a TOF of the coincidence event if the corresponding photons were detected by the two corrected scintillator elements. The simulated TOF may be designated as the corrected TOF. Exemplary Monte-Carlo simulation tools may include an Electron Gamma Shower (EGSnrc), a Monte-Carlo N-Particle Transport (MCNP), a GEometry ANd Tracking (Geant4), a Dose Planning Method (DPM) tool, a Voxel-based Monte Carlo (VMC), a VMC++, or any other simulation tools designed according to the Monte-Carlo simulation algorithm, or any combination thereof.

In some embodiments, the processing device 140 may determine the corrected TOF of each coincidence event according to an experimental TOF result. For example, experiments may be done to obtain an experimental time difference between a first TOF and a second TOF under same experimental conditions (e.g., the positron and the electron annihilated at a center of the detector 112). The first TOF may be obtained when the positron and the electron annihilated at a center of the detector 112 (e.g., the center of the field of view (FOV) of the detector 112, a point on the long axis of the detector 112) and the two photons generated from the annihilation were detected by the two original scintillator elements. The second TOF may be obtained when positron and the electron annihilated at a center of the detector 112 (e.g., the center of the field of view (FOV) of the detector 112, a point on the long axis of the detector 112) and the two photons generated from the annihilation were detected by the two corrected scintillator elements. The processing device 140 may determine the corrected TOF by subtracting the experimental time difference from an original TOF that were measured when two photons of a coincidence event of the plurality of coincidence events were detected by the two original scintillator elements and the identities of the two original scintillator elements are corrected by the identities of the two corrected scintillator elements. The original TOF may be obtained when the positron and the electron annihilated at any location of the detector 112. Techniques for identifying the original TOF of each coincidence event may be found in, e.g., U.S. Pat. No. 8,164,063B2, the contents of which are hereby incorporated by reference. As another example, experiments may be done to obtain a table including a plurality of experimental time differences. Each of the plurality of experimental time differences corresponds to a layer count difference (e.g., one layer indicating that a difference between a first layer count (e.g., 3) and a second layer count (e.g., 2) is 1, a first photon of an experimental coincidence event was detected by a scintillator element at the 3^{rd} layer of the scintillator element arrays and a second photon of the experimental coincidence event was detected by a scintillator element at a 2^{nd} layer of the scintillator element arrays). As used herein, the scintillator element arrays may include a plurality of layers of scintillator elements along the incidence direction of the photons. The processing device 140 may retrieve the table using a total layer difference between each original scintillator element and the corresponding corrected scintillator element. For example, the total layer difference of a coincidence event is 3 (3=2+1, where “2” indicates that a layer difference between a first original scintillator element and the corresponding first corrected scintillator element of the coincidence event is 2, and “1” indicates that a layer difference between a second original scintillator element and the corresponding second corrected scintillator element is 1). The processing device 140 may retrieve the table using the total layer difference and obtain an experimental time difference corresponding to the total layer difference. The processing device 140 may determine the corrected TOF by subtracting the experimental time difference from the original TOF.

In some embodiments, the processing device 140 may store the corrected TOF corresponding to each of the plurality of coincidence events in corrected list-mode data, a corrected sinogram, a corrected histogram, or the like, or any combination thereof. For example, to provide the corrected list-mode data (or the corrected sinogram), the processing device 140 may replace the original TOF with the corrected TOF of each coincidence event in the original list-mode data (or the original sinogram), in addition to or instead of replacing an identity of an original scintillator element of each coincidence event with an identity of a corrected scintillator element corresponding to the original scintillator element in original list-mode data (or an original sinogram), and/or replacing a DOI of a photon of each coincidence event with a corrected DOI (e.g., 0) of the photon in original list-mode data (or original sinogram).

Merely by way of example, the corrected list-mode data (or the corrected sinogram) may be generated by replacing the original TOF with the corrected TOF of each coincidence event in the original list-mode data (or the original sinogram). As another example, the corrected list-mode data (or the corrected sinogram) may be generated by replacing the original TOF with the corrected TOF of each coincidence event in the original list-mode data (or the original sinogram), and also replacing an identity of an original scintillator element of each coincidence event with an identity of a corrected scintillator element corresponding to the original scintillator element in original list-mode data (or an original sinogram). As a further example, the corrected list-mode data (or the corrected sinogram) may be generated by replacing the original TOF with the corrected TOF of each coincidence event in the original list-mode data (or the original sinogram), also replacing an identity of an original scintillator element of each coincidence event with an identity of a corrected scintillator element corresponding to the original scintillator element in original list-mode data (or an original sinogram), and further replacing a DOI of a photon of each coincidence event with a corrected DOI (e.g., 0) of the photon in original list-mode data (or original sinogram). As still a further example, the corrected list-mode data (or the corrected sinogram) may be generated by replacing an identity of an original scintillator element of each coincidence event with an identity of a corrected scintillator element corresponding to the original scintillator element in original list-mode data (or an original sinogram). As still a further example, the corrected list-mode data (or the corrected sinogram) may be generated by replacing an identity of an original scintillator element of each coincidence event with an identity of a corrected scintillator element corresponding to the original scintillator element in original list-mode data (or an original sinogram), and further replacing a DOI of a photon of each coincidence event with a corrected DOI (e.g., 0) of the photon in original list-mode data (or original sinogram).

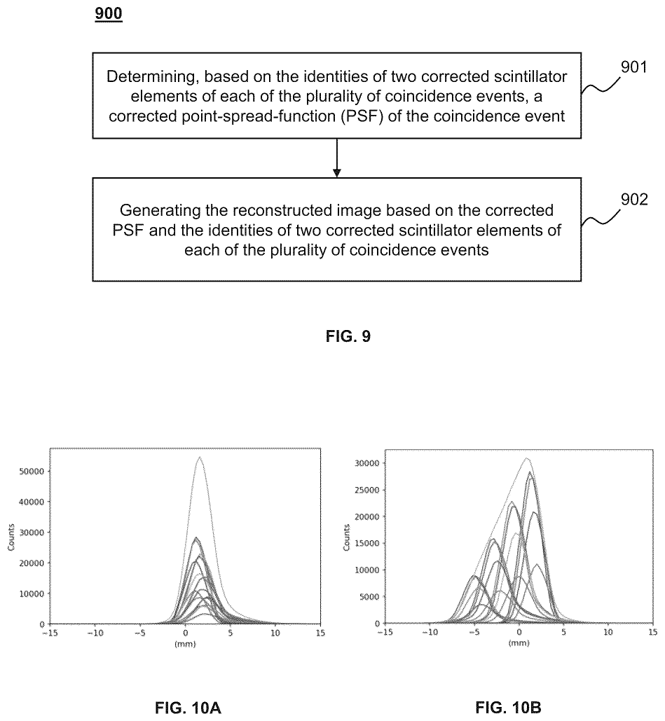

In some embodiments, the processing device 140 may generate the reconstructed image based on the corrected list-mode data (or the corrected sinogram, the corrected histogram). In some embodiments, the processing device 140 may perform one or more operations in process 900 as described in connection with FIG. 9 to generate the reconstructed image.

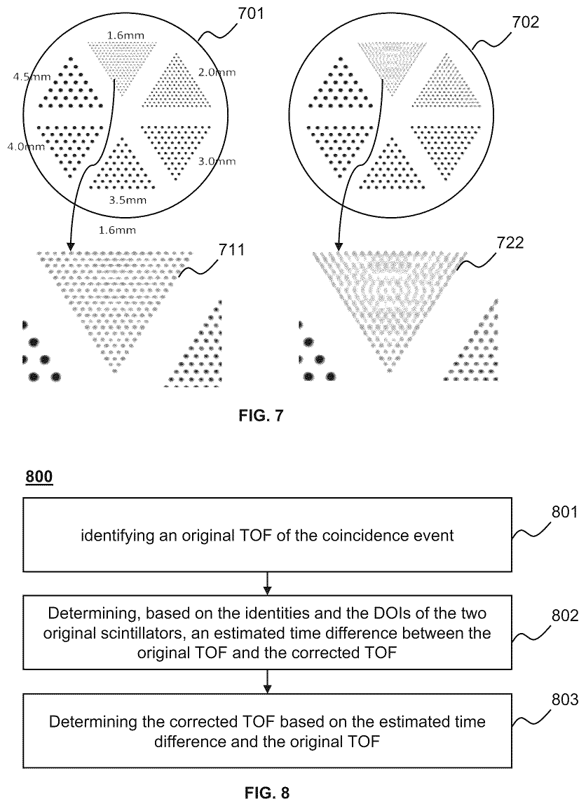

FIG. 7 illustrates exemplary reconstructed images of a plurality of rods according to some embodiments of the present disclosure. As shown in FIG. 7, a reconstructed image 701 of the plurality of rods was generated based on the identities of two corrected scintillator elements of each coincidence event, and a reconstructed image 702 was generated based on the identities of two original scintillator elements of each coincidence event whiling ignoring the effects of non-zero DOIs. The reconstructed image 701 and the reconstructed image 702 show rods of different diameters, such as 1.6 mm (shown in enlarged views 711 and 722, respectively), 2.0 mm, 3.0 mm, 3.5 mm, 4.0 mm, and 4.5 mm, respectively. As shown in enlarged views 711 and 722 of FIG. 7, the 1.6 mm rods can be distinguished near a field of view (FOV) center (shown in the center of the reconstructed image 701 and the reconstructed image 702). However, the resolution at off-center locations of the reconstructed image 702 degrades significantly due to the effects of the ignored non-zero DOIs. The 1.6 mm rods are distinguishable even at off-center locations of the reconstructed image 701.

According to the embodiments of the present disclosure, based on the identities and the DOIs of the two original scintillator elements of each coincidence event of the plurality of coincidence events, the systems and methods may determine the identities of two corrected scintillator elements each of which corresponds to an original scintillator element and/or the corrected DOI, and/or the corrected TOF of the coincidence event for image reconstruction. The systems and methods, when performing image reconstruction, may take into consideration the non-zero DOIs by correcting information relating to the plurality of coincidence events in the list-mode data (or the sinogram) while avoiding modification of existing reconstruction algorithms, thereby improving image resolutions without increasing reconstruction complexity.

It should be noted that the above description regarding the process 500 is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure. In some embodiments, one or more operations may be added or omitted. For example, the process 500 may include an additional operation to transmit the reconstructed image to a terminal (e.g., a terminal 130) for display. As another example, in 504, for each of the plurality of coincidence events, when the processing device 140 (e.g., the DOI correction module 420) obtains the identities and/or the DOIs of two original scintillator elements that have detected photons of a coincident event, the processing device 140 may first determine whether either one of the two original scintillator elements are located in the photon incident surface of the scintillator element arrays (or the detector 112) or if either one of the DOIs of the two photons is zero. For a photon that has been detected by an original scintillator element located in the photon incident surface of the scintillator element arrays (or the detector 112) or whose DOI is zero, the original scintillator element may be directly designated as the corrected scintillator element with respect to the photon.

FIG. 8 is a flowchart illustrating an exemplary process 800 for determining a corrected TOF according to some embodiments of the present disclosure. In some embodiments, the process 800 may be executed by the imaging system 100. For example, the process 800 may be implemented as a set of instructions (e.g., an application) stored in a storage device (e.g., the storage device 150, the storage device 220, and/or the storage 390). In some embodiments, the processing device 140 (e.g., the processor 210 of the computing device 200, the CPU 340 of the mobile device 300, and/or one or more modules illustrated in FIG. 4) may execute the set of instructions and may accordingly be directed to perform the process 800. In some embodiments, one or more operations of the process 800 may be performed to achieve at least part of operation 505 as described in connection with FIG. 5.

In 801, for each of the plurality of coincidence events, the processing device 140 (e.g., the TOF correction module 430) may identify an original TOF of the coincidence event. In some embodiments, the processing device 140 may extract the original TOF of the coincidence event from the information of the coincidence event.

In 802, the processing device 140 (e.g., the TOF correction module 430) may determine, based on the identities and the DOIs of the two original scintillator elements, an estimated time difference between the original TOF and the corrected TOF.

In some embodiments, the estimated time difference may be used to compensate for a time difference caused by correcting the identities of the two original scintillator elements using the identities of the two corrected scintillator elements. For example, the processing device 140 may determine the estimated time difference according to Equations (1)-(3):

Δt=Δtₐ−Δt_{b}, (1)

Δtₐ=−lₐ/cₕ+dₐ/cᵢ, (2)

and

Δt_{b}−l_{b}/cₕ+d_{b}/cᵢ, (3)

where Δt denotes an estimated time difference of a coincidence event, Δtₐ denotes a first estimated time difference component corresponding to a first photon of the coincidence event, Δt_{b} denotes a second estimated time difference component corresponding to a second photon of the coincidence event, lₐ denotes an actual flying distance of the first photon, dₐ denotes a DOI of the first photon, l_{b} denotes an actual flying distance of the second photon, d_{b} denotes a DOI of the second photon, cₕ denotes a photon speed in a first medium (e.g., air), and cᵢ denotes a photon speed in a second medium (e.g., the scintillator crystal). In some embodiments, the photon speed cₕ in the first medium (e.g., air) may be the speed of light. The photon speed in the second medium (e.g., the scintillator crystal) may be determined based on a refractive index of the second medium (e.g., the scintillator crystal) or measured experimentally.

In some embodiments, the actual flying distance lₐ of the first photon may be determined based on the identity of the first original scintillator element of the two original scintillator elements and the DOI of the first photon. For example, as shown in FIG. 6, an incident angle of the first photon may be determined based on the identity Oa of the first original scintillator element, the DOI da of the first photon, the identity Ob of the second original scintillator element, and the DOI db of the second photon. The actual flying distance lₐ of the first photon may be determined based on the incident angle of the first photon and the DOI da of the first photon according to a trigonometric function algorithm. The actual flying distance l_{b} of the second photon may be determined according to a same process for determining the actual flying distance lₐ of the first photon.

In 803, the processing device 140 (e.g., the TOF correction module 430) may determine the corrected TOF based on the estimated time difference and the original TOF. In some embodiments, the processing device 140 may determine the corrected TOF by subtracting the estimated time difference from the original TOF.

It should be noted that the above description regarding the process 800 is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure. In some embodiments, one or more operations may be added or omitted. For example, the process 800 may include an additional operation to store the corrected TOF in a corrected list-mode data or a corrected sinogram.

FIG. 9 is a flowchart illustrating an exemplary process 900 for generating a reconstructed image according to some embodiments of the present disclosure. In some embodiments, the process 900 may be executed by the imaging system 100. For example, the process 900 may be implemented as a set of instructions (e.g., an application) stored in a storage device (e.g., the storage device 150, the storage device 220, and/or the storage 390). In some embodiments, the processing device 140 (e.g., the processor 210 of the computing device 200, the CPU 340 of the mobile device 300, and/or one or more modules illustrated in FIG. 4) may execute the set of instructions and may accordingly be directed to perform the process 900. In some embodiments, one or more operations of the process 900 may be performed to achieve at least part of operation 505 as described in connection with FIG. 5.

In 901, the processing device 140 (e.g., the reconstruction module 440) may determine, based on the identities of two corrected scintillator elements of each of the plurality of coincidence events, a corrected point-spread-function (PSF) of the coincidence event.

In some embodiments, the processing device 140 may determine the corrected PSF of the coincidence event according to a PSF simulation algorithm. For example, the processing device 140 may determine the corrected PSF by applying a Monte-Carlo Simulation algorithm on the identities of two corrected scintillator elements of each of the plurality of coincidence events (and/or the corrected DOIs and/or the corrected TOF). For example, the processing device 140 may utilize a Monte-Carlo simulation tool (e.g., a software, an application, a toolkit) to simulate an PSF of the coincidence event if the corresponding photons were detected by the two corrected scintillator elements. The simulated PSF may be designated as the corrected PSF. Exemplary Monte-Carlo simulation tools may include an Electron Gamma Shower (EGSnrc), a Monte-Carlo N-Particle Transport (MCNP), a GEometry ANd Tracking (Geant4), a Dose Planning Method (DPM) tool, a Voxel-based Monte Carlo (VMC), a VMC++, or any other simulation tools designed according to the Monte-Carlo simulation algorithm, or any combination thereof. In some embodiments, the corrected PSF of the coincidence event may be determined according to an experimental PSF result. For example, experiments may be done to obtain an experimental PSF result when the corresponding photons were detected by the two corrected scintillator elements.

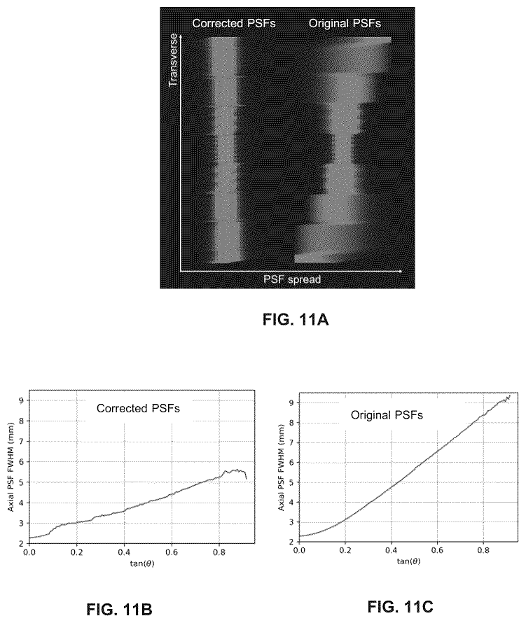

FIG. 10A illustrates exemplary corrected PSFs according to some embodiments of the present disclosure. FIG. 10B illustrates exemplary original PSFs according to some embodiments of the present disclosure. As shown in FIG. 10A and FIG. 10B, a full-width-half-maximum (FWHM) of a corrected PSF is smaller than that of an original PSF at a same off-center location. FIG. 11A illustrates FWHMs of exemplary corrected PSFs and exemplary original PSFs along a transverse direction according to some embodiments of the present disclosure. FIG. 11B illustrates FWHMs of exemplary corrected PSFs along an axial direction according to some embodiments of the present disclosure. FIG. 11C illustrates FWHMs of exemplary original PSFs along an axial direction according to some embodiments of the present disclosure. In some embodiments, the more DOI effects (corresponding to the more significantly the DOIs deviating from zero), the wider an FWHM of a PSF, and the poorer the image resolution of a reconstructed image may be. Thus, compared to wide FWHMs, narrow FWHMs of the corrected PSFs may result in a high image resolution of the reconstructed image. According to FIGS. 10A, 10B and 11A, minimal DOI effects may be observed near the FOV center, and a significant increase of FWHM may be observed at off-center locations. According to FIGS. 11B and 11C, the FWHMs of corrected PSFs may be narrower than the FWHMs of original PSFs at a same incident angle. However, due to inter-crystal scatter, the FWHMs of corrected PSFs at incident angles other than 0 are still wider than that of a PSF (or a corrected PSF) at an incident angle of 0.

In 902, the processing device 140 (e.g., the reconstruction module 440) may generate the reconstructed image based on the corrected PSF and the identities of two corrected scintillator elements of each of the plurality of coincidence events.

In some embodiments, the processing device 140 may generate the reconstructed image based on the corrected PSF and the identities of two corrected scintillator elements of each of the plurality of coincidence events according to a reconstruction algorithm. Alternatively, the processing device 140 may generate the reconstructed image based on the corrected PSF, the identities of two corrected scintillator elements, the corrected DOI, and/or the corrected TOF (or in the format of the corrected list-mode data or the corrected sinogram) of each of the plurality of coincidence events according to the reconstruction algorithm. Exemplary reconstruction algorithms may include an expectation maximization (EM) algorithm, a maximum likelihood expectation maximization (MLEM) algorithm, a complete ordered subset expectation maximization (C-OSEM) algorithm, a filter back projection (FBP) algorithm, or the like, or any combination thereof.

It should be noted that the above description regarding the process 900 is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure. In some embodiments, one or more operations may be added or omitted. For example, the process 900 may include an additional operation to transmit the reconstructed image to a terminal (e.g., a terminal 130) for display.

It will be apparent to those skilled in the art that various changes and modifications can be made in the present disclosure without departing from the spirit and scope of the disclosure. In this manner, the present disclosure may be intended to include such modifications and variations if the modifications and variations of the present disclosure are within the scope of the appended claims and the equivalents thereof.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C #, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification is provided for illustration purposes and modified according to different situations. For example, a classification condition that “a probability value is greater than the threshold value” may further include or exclude a condition that “the probability value is equal to the threshold value”.

1. A system, comprising:
at least one storage device including a set of instructions; and
at least one processor in communication with the at least one storage device, wherein when executing the set of instructions, the at least one processor is directed to cause the system to perform operations including:
obtaining image data that includes information of a plurality of coincidence events;
for each of the plurality of coincidence events,
identifying, based on the information of the coincidence event, identities of two original scintillator elements at each of which a photon of the coincidence event was detected;
obtaining a depth of interaction (DOI) of each photon of the coincidence event within one original scintillator element of the two original scintillator elements; and
determining, based on the identities and the DOIs of the two original scintillator elements, identities of two corrected scintillator elements each of which corresponds to an original scintillator element of the two original scintillator elements; and
generating a reconstructed image based on the identities of two corrected scintillator elements of each of the plurality of coincidence events.

2. The system of claim 1, wherein for each of the plurality of coincidence events, a corrected DOI of each photon of the coincidence event in the corresponding corrected scintillator element is a pre-determined value.

3. The system of claim 2, wherein the operations further include:
storing the identities of two corrected scintillator elements corresponding to each of the plurality of coincidence events into corrected list-mode data or a corrected sinogram; and
generating the reconstructed image based on the corrected list-mode data or the corrected sinogram.

4. The system of claim 1, wherein the determining, based on the identities and the DOIs of the two original scintillator elements, the identities of the two corrected scintillator elements includes:
determining, based on the identities and the DOIs of the two original scintillator elements, a projection line of the coincidence event;
determining two intersection points of the projection line and a scintillator element array; and
determining the identities of the two corrected scintillator elements based on the two intersection points.

5. The system of claim 1, wherein the generating the reconstructed image based on the identities of the two corrected scintillator elements includes:
determining a corrected time of flight (TOF) for each of the plurality of coincidence events; and
generating the reconstructed image based on the identities of two corrected scintillator elements and the corrected TOF of each of the plurality of coincidence events.

6. The system of claim 5, wherein the determining the corrected TOF includes:
identifying an original TOF of the coincidence event;
determining, based on the identities and the DOIs of the two original scintillator elements, an estimated time difference between the original TOF and the corrected TOF; and
determining the corrected TOF based on the estimated time difference and the original TOF.

7. The system of claim 5, wherein the corrected TOF is determined according to a TOF simulation algorithm or an experimental TOF result.

8. The system of claim 5, wherein the operations further include:
storing the corrected TOF into corrected list-mode data or a corrected sinogram; and
generating the reconstructed image based on the corrected list-mode data or the corrected sinogram.

9. The system of claim 1, wherein the generating the reconstructed image based on the identities of the two corrected scintillator elements of each of the plurality of coincidence events includes:
determining, based on the identities of two corrected scintillator elements of each of the plurality of coincidence events, a corrected point-spread-function (PSF) of the coincidence event; and
generating the reconstructed image based on the corrected PSF and the identities of two corrected scintillator elements of each of the plurality of coincidence events.

10. The system of claim 9, wherein the corrected PSF is determined according to a PSF simulation algorithm or an experimental PSF result.

11. A method, the method being implemented on a computing device having at least one storage device and at least one processor, the method comprising:
obtaining image data that includes information of a plurality of coincidence events;
for each of the plurality of coincidence events,
identifying, based on the information of the coincidence event, identities of two original scintillator elements at each of which a photon of the coincidence event was detected;
obtaining a depth of interaction (DOI) of each photon of the coincidence event within one original scintillator element of the two original scintillator elements; and
determining, based on the identities and the DOIs of the two original scintillator elements, identities of two corrected scintillator elements each of which corresponds to an original scintillator element of the two original scintillator elements; and
generating a reconstructed image based on the identities of two corrected scintillator elements of each of the plurality of coincidence events.

12. The method of claim 11, wherein for each of the plurality of coincidence events, a corrected DOI of each photon of the coincidence event in the corresponding corrected scintillator element is a pre-determined value.

13. The method of claim 12, further comprising:
storing the identities of two corrected scintillator elements corresponding to each of the plurality of coincidence events into corrected list-mode data or a corrected sinogram; and
generating the reconstructed image based on the corrected list-mode data or the corrected sinogram.

14. The method of claim 11, wherein the determining, based on the identities and the DOIs of the two original scintillator elements, the identities of the two corrected scintillator elements includes:
determining, based on the identities and the DOIs of the two original scintillator elements, a projection line of the coincidence event;
determining two intersection points of the projection line and a scintillator element array; and
determining the identities of the two corrected scintillator elements based on the two intersection points.

15. The method of claim 11, wherein the generating the reconstructed image based on the identities of the two corrected scintillator elements includes:
determining a corrected time of flight (TOF) for each of the plurality of coincidence events; and
generating the reconstructed image based on the identities of two corrected scintillator elements and the corrected TOF of each of the plurality of coincidence events.

16. The method of claim 15, wherein the determining the corrected TOF includes:
identifying an original TOF of the coincidence event;
determining, based on the identities and the DOIs of the two original scintillator elements, an estimated time difference between the original TOF and the corrected TOF; and
determining the corrected TOF based on the estimated time difference and the original TOF.

17. The method of claim 15, wherein the corrected TOF is determined according to a TOF simulation algorithm or an experimental TOF result.

18. The method of claim 15, further comprising:
storing the corrected TOF into corrected list-mode data or a corrected sinogram; and
generating the reconstructed image based on the corrected list-mode data or the corrected sinogram.

19. The method of claim 11, wherein the generating the reconstructed image based on the identities of the two corrected scintillator elements of each of the plurality of coincidence events includes:
determining, based on the identities of two corrected scintillator elements of each of the plurality of coincidence events, a corrected point-spread-function (PSF) of the coincidence event; and
generating the reconstructed image based on the corrected PSF and the identities of two corrected scintillator elements of each of the plurality of coincidence events.

20. (canceled)

21. A non-transitory computer readable medium, comprising at least one set of instructions, wherein when executed by at least one processor of a computing device, the at least one set of instructions causes the computing device to perform a method, the method comprising:
obtaining image data that includes information of a plurality of coincidence events;
for each of the plurality of coincidence events,
identifying, based on the information of the coincidence event, identities of two original scintillator elements at each of which a photon of the coincidence event was detected;
obtaining a depth of interaction (DOI) of each photon of the coincidence event within one original scintillator element of the two original scintillator elements; and
determining, based on the identities and the DOIs of the two original scintillator elements, identities of two corrected scintillator elements each of which corresponds to an original scintillator element of the two original scintillator elements; and
generating a reconstructed image based on the identities of two corrected scintillator elements of each of the plurality of coincidence events.
